(19) United States
(12) Patent Application Publication
(10) Pub. No.: US 2026/0115330 A1
(43) Pub. Date: Apr. 30, 2026

(54) RADIOPAQUE MULTI-ARM POLYMERS FOR MEDICAL APPLICATIONS

(71) Applicant: Boston Scientific Scimed, Inc., Maple Grove, MN (US)

(72) Inventors: Rick William Dorn, Plymouth, MN (US); Joseph Thomas Delaney, JR., Minneapolis, MN (US); Cristian Parisi, Amherst, MA (US)

(73) Assignee: Boston Scientific Scimed, Inc., Maple Grove, MN (US)

(21) Appl. No.: 19/374,350

(22) Filed: Oct. 30, 2025

Related U.S. Application Data
(60) Provisional application No. 63/714,657, filed on Oct. 31, 2024.

(51) Int. Cl. A61K 49/04

(52) U.S. Cl. CPC A61K 49/0442

(57) ABSTRACT

In some aspects, the present disclosure pertains to multi-arm polymers that comprise a core region, a plurality of polymer arms linked to the core region, at least three of the polymer arms each comprising a hydrophilic polymer segment and an amino-alcohol residue that is attached to the hydrophilic polymer segment through an ether group, an electrophilic group attached to each amino-alcohol residue through a linkage that comprises a hydrolysable ester group, and a residue of a carboxylic-acid-containing iodinated molecule attached to each amino-alcohol residue through a linkage comprising an amide group. Other aspects of the present disclosure pertain to systems for forming hydrogels that are based on such multi-arm polymers, to crosslinked products that are formed from such systems, and to methods of treatment that use such systems.

### CROSS-REFERENCE TO RELATED APPLICATIONS

This application claims the benefit of U.S. Provisional Patent Application Ser. No. 63/714,657 filed on Oct. 31, 2024, the disclosure of which is incorporated herein by reference.

### FIELD

The present disclosure relates to radiopaque multi-arm polymers, to methods of making such multi-arm polymers, to crosslinkable systems for radiopaque hydrolysable hydrogels, and to methods of treatment using such radiopaque hydrolysable hydrogels.

### BACKGROUND

SpaceOAR®, a rapid crosslinking hydrogel that polymerizes in vivo within seconds, is based on a multi-arm polyethylene glycol (PEG) polymer with a polyol core functionalized with succinimidyl glutarate as reactive end groups which further react with trilysine to form crosslinks. This product has become a very successful, clinically used biomaterial in prostate cancer therapy. A further improvement based on this structure is that a portion of the succinimidyl glutarate end groups have been replaced with 2,3,5-triiiodobenzamide groups, providing radiopacity. This hydrogel, known by the trade name of SpaceOAR Vue®, is the radiopaque version of SpaceOAR® for prostate medical applications. Above a specific pH, the succinimidyl glutarate groups of SpaceOAR® and SpaceOAR Vue® will rapidly react with the trilysine crosslinker in vivo to form a hydrogel. The hydrogel breaks down in-vivo over the course of about 6-9 months. The breakdown occurs primarily through the hydrolysis of the ester linkages in the glutarate groups.

An alternative approach to the synthesis of an iodinated multi-arm polymer is described herein, which enables water-soluble iodinated species to be provided in some or all reactive arms of the multi-arm polymer.

### SUMMARY

In some aspects, the present disclosure pertains to a multi-arm polymer comprising a core region, a plurality of polymer arms linked to the core region, at least three of the polymer arms each comprising a hydrophilic polymer segment and an amino-alcohol residue that is attached to the hydrophilic polymer segment through an ether group, an electrophilic group attached to each amino-alcohol residue through a linkage that comprises a hydrolysable ester group, and a residue of a carboxylic-acid-containing iodinated molecule attached to each amino-alcohol residue through a linkage comprising an amide group.

In some embodiments, the amino-alcohol residue comprises an amino group and a hydroxyl group on adjacent carbon atoms.

In some embodiments, which can be used in conjunction with the above aspects and embodiments, the electrophilic group is a cyclic-imidyl ester group.

In some embodiments, which can be used in conjunction with the above aspects and embodiments, the carboxylic-acid-containing iodinated molecule comprises a carboxylic acid group and a monocyclic or multicyclic aromatic structure that is substituted with one or more iodine atoms.

In some embodiments, which can be used in conjunction with the above aspects and embodiments, the carboxylic-acid-containing iodinated molecule is a carboxyalkylamido-modified, iodinated amino-acid ester, and wherein a residue of an iodinated amino-acid ester is attached to the amino-alcohol residue through a linkage that contains two amide groups.

In some embodiments, which can be used in conjunction with the above aspects and embodiments, the multi-arm polymer is made by a process comprising (a) reacting the carboxylic-acid-containing iodinated molecule in an amide coupling reaction with a multi-arm polymer comprising the core region and a plurality of polymer arms linked to the core region, at least three of the polymer arms each comprising the hydrophilic polymer segment and an amino-alcohol end group that is attached to the hydrophilic polymer segment through an ether group; (b) reacting the product of step (a) with a cyclic anhydride compound in a ring opening reaction; (c) reacting the product of step (b) in an ester-forming reaction with an N-hydroxy cyclic imide compound. In some of these embodiments, the carboxylic-acid-containing iodinated molecule is made by a process that comprises reacting a cyclic anhydride compound in a ring opening reaction with an amino group of an iodinated amino-acid alkyl ester to form a carboxyalkylamido-modified iodinated amino-acid alkyl ester.

In some aspects, the present disclosure pertains to a multi-arm polymer comprising a core region, a plurality of polymer arms linked to the core region, at least three of the polymer arms each comprising a hydrophilic polymer segment and a residue of a halogen-substituted cyclic anhydride that is attached to the hydrophilic polymer segment through a hydrolysable ester group, an electrophilic group attached to each halogen-substituted cyclic anhydride residue, and at least one residue of a hydroxyl-containing iodinated molecule attached to each halogen-substituted cyclic anhydride residue through a linkage comprising an ether group, wherein the halogen is selected from bromine and iodine.

In some embodiments, the halogen-substituted cyclic anhydride comprises two or more halogen groups.

In some embodiments, which can be used in conjunction with the above aspects and embodiments, the electrophilic group is a cyclic-imidyl ester group that is directly attached to a carbon atom of each halogen-substituted cyclic anhydride residue.

In some embodiments, which can be used in conjunction with the above aspects and embodiments, the hydroxyl-containing iodinated molecule comprises a hydroxyl group and a monocyclic or multicyclic aromatic structure that is substituted with one or more iodine atoms.

In some embodiments, which can be used in conjunction with the above aspects and embodiments, the multi-arm polymer is made by a process comprising (a) reacting a hydroxyl-terminated multi-arm polymer, which comprises the core region and a plurality of polymer arms linked to the core region, at least three of the polymer arms each comprising the hydrophilic polymer segment and a hydroxyl end group, with a halogen-substituted cyclic anhydride compound to form a multi-arm polymer, which comprises the core region and a plurality of polymer arms linked to the core region, at least three of the polymer arms each comprising the hydrophilic polymer segment and a carboxyhaloalkyl end group that is attached to the hydrophilic polymer segment through a hydrolysable ester group; (b) reacting the product of step (a) with at least one hydroxyl-containing iodinated molecule in a substitution reaction to form a multi-arm polymer, which comprises the core region and a plurality of polymer arms linked to the core region, at least three of the polymer arms each comprising the hydrophilic polymer segment and at least one residue of the hydroxyl-containing iodinated molecule attached to a residue of the carboxyhaloalkyl end group through a linkage comprising an ether group; and (c) reacting the product of step (b) in an ester-forming reaction with an N-hydroxy cyclic imide compound.

In some aspects, the present disclosure pertains to a multi-arm polymer comprising a core region, a plurality of polymer arms linked to the core region, at least three of the polymer arms each comprising a hydrophilic polymer segment and a residue of a carboxylic-acid-substituted cyclic anhydride that is attached to the hydrophilic polymer segment through a hydrolysable ester group, a residue of a hydroxyl-containing iodinated molecule attached to each carboxylic-acid-substituted cyclic anhydride residue through a linkage that comprises a urethane group and an amide group, and an electrophilic group attached to each carboxylic-acid-substituted cyclic anhydride residue.

In some embodiments, the electrophilic group is a cyclic-imidyl ester group that is directly attached to a carbon atom of each carboxylic-acid-substituted cyclic anhydride residue.

In some embodiments, which can be used in conjunction with the above aspects and embodiments, the linkage that comprises a urethane group and an amide group contains a residue of compound that comprises an amine group and an isocyanate group.

In some embodiments, which can be used in conjunction with the above aspects and embodiments, the carboxylic-acid-substituted cyclic anhydride comprises two or more carboxylic acid groups.

In some embodiments, which can be used in conjunction with the above aspects and embodiments, the hydroxyl-containing iodinated molecule comprises a hydroxyl group and a monocyclic or multicyclic aromatic structure that is substituted with one or more iodine atoms.

In some embodiments, which can be used in conjunction with the above aspects and embodiments, the multi-arm polymer is made by a process comprising (a) reacting a hydroxyl-terminated multi-arm polymer, which comprises the core region and a plurality of polymer arms linked to the core region, at least three of the polymer arms each comprising the hydrophilic polymer segment and a hydroxyl end group, in a ring opening reaction with carboxylic-acid-substituted cyclic anhydride compound, in which the carboxylic acid is protected, to form a multi-arm polymer that comprises the core region and a plurality of polymer arms linked to the core region, at least three of the polymer arms each comprising the hydrophilic polymer segment and an end group that comprises the protected carboxylic acid group and an unprotected protected carboxylic acid group; (b) reacting a hydroxyl-containing iodinated molecule with a compound that comprises a protected amine group and an isocyanate group and then deprotecting the amine group to form a molecule in which an amine group is attached to a residue of the hydroxyl-containing iodinated molecule through a urea-containing linkage; (c) reacting the product of step (a) with the product of step (b) in an amide coupling reaction; (d) deprotecting the protected carboxylic acid group; and (e) reacting the product of step (d) in an ester-forming reaction with an N-hydroxy cyclic imide compound.

In other aspects, the present disclosure pertains to a system for forming a hydrogel that comprises (a) a multi-arm polymer in accordance with the any of the above aspects and embodiments and (b) a polyamino compound comprising at least two amino (—NH₂) groups, wherein the reactive multi-arm polymer and the polyamino compound react to form a crosslinked hydrogel.

In additional aspects, the present disclosure pertains to a medical hydrogel formed by crosslinking the multi-arm polymer in accordance with the any of the above aspects and embodiments and a polyamino compound that forms covalent crosslinks with the multi-arm polymer.

In further aspects, the present disclosure pertains to a method of treatment comprising administering to a subject a mixture that comprises the multi-arm polymer in accordance with the any of the above aspects and embodiments and a polyamino compound under conditions such that the multi-arm polymer and the polyamino compound form covalent crosslinks after administration.

Potential benefits associated with the present disclosure include one or more of the following: higher radiopacity, consistent water solubility properties, and the ability of all arms of the multi-arm polymer to be able to react with a crosslinking agent.

The above and other aspects, embodiments, features and benefits of the present disclosure will be readily apparent from the following detailed description.

### BRIEF DESCRIPTION OF THE DRAWINGS

FIG. 1 schematically illustrates the formation of reactive multi-arm polymer, in accordance with an embodiment of the present disclosure.

FIG. 2 schematically illustrates the formation of reactive multi-arm polymer, in accordance with another embodiment of the present disclosure.

FIG. 3 schematically illustrates the formation of reactive multi-arm polymer, in accordance with an additional embodiment of the present disclosure.

FIG. 4 schematically illustrates the formation of reactive multi-arm polymer, in accordance with a further embodiment of the present disclosure.

FIG. 5 schematically illustrates a crosslinking reaction, in accordance with a further embodiment of the present disclosure.

FIG. 6 schematically illustrates a delivery device, in accordance with an embodiment of the present disclosure.

FIG. 7 schematically illustrates a delivery device, in accordance with another embodiment of the present disclosure.

### DETAILED DESCRIPTION

In some aspects, the present disclosure pertains to radiopaque reactive polymers comprising reactive moieties that are reactive with amino groups of polyamino compounds.

In some aspects, the present disclosure pertains to radiopaque hydrolysable hydrogels are provided that comprise crosslinked reaction products of radiopaque reactive polymers and polyamino compounds.

As used herein, a “hydrogel” is a crosslinked polymer that contains water or can absorb water but does not dissolve when placed in water.

Radiopaque reactive polymers for use in the present disclosure include reactive multi-arm polymers that comprise a core region, a plurality of polymer arms linked to the core region, at least three of the polymer arms each comprising a hydrophilic polymer segment and an amino-alcohol residue that is attached to the hydrophilic polymer segment through an ether group, an electrophilic group attached to each amino-alcohol residue through a linkage that comprises a hydrolysable ester group, and a residue of a carboxylic-acid-containing iodinated molecule attached to each amino-alcohol residue through a linkage comprising an amide group.

Radiopaque multi-arm reactive polymers in accordance with the present disclosure include polymers having from 3 to 100 arms, for example ranging anywhere from 3 to 4 to 5 to 6 to 7 to 8 to 10 to 12 to 15 to 20 to 25 to 50 to 75 to 100 arms.

Hydrophilic polymer segments for the polymer arms can be selected from any of a variety of synthetic, natural, or hybrid synthetic-natural polymer segments. Examples of polymer segments include those that are formed from one or more monomers selected from the following: C₁-C₆-alkylene oxides (e.g., ethylene oxide, propylene oxide, tetramethylene oxide, etc.), cyclic ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, poly(N-isopropylacrylamide) segments, polypeptide segments, and polysaccharide segments.

Polymer segments for use in the multi-arm polymers of the present disclosure typically contain between 5 and 1000 monomer units or more, for examples ranging anywhere from 5 to 10 to 25 to 50 to 100 to 250 to 500 to 1000 monomer units (i.e., ranging between any two of the preceding values).

Electrophilic groups may be selected, for example, from cyclic imide ester groups (also referred to as cyclic-imidyl-oxycarbonyl groups), such as succinimide ester groups,

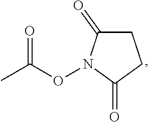

maleimide ester groups, glutarimide ester groups, diglycolimide ester groups, phthalimide ester groups, bicyclo[2.2.1]hept-5-ene-2,3-dicarboxylic acid imide ester groups,

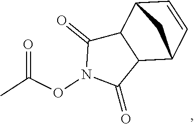 [SMILES: CC(=O)ON1C(=O)C2C(C1=O)[C@H]1C=C[C@@H]2C1]

and tetrahydro-1H-azepine-2,7-dione ester groups,

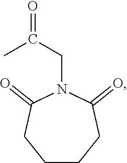

among other possibilities.

Core regions for use in the present disclosure include core regions that comprise a residue of a polyol comprising three or more hydroxyl groups, which is used to form the polymer arms. In certain beneficial embodiments, the core region comprises a residue of a polyol that contains from 3 to 100 hydroxyl groups, for example ranging anywhere from 3 to 4 to 5 to 6 to 7 to 8 to 9 to 10 to 11 to 12 to 15 to 20 to 25 to 50 to 75 to 100 hydroxy groups.

Illustrative polyols may be selected, for example, from sugars (monosaccharides, disaccharides, trisaccharides, etc.), sugar alcohols, calixarenes, cyclodextrins, polyhydroxylated polymers, catechins, flavanols, anthocyanins, stilbenes, and polyphenols, among others.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols. Specific examples include methane triol, glycerol, trimethylolpropane, benzenetriol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, catechins, flavanols, anthocyanins, stilbenes, polyphenols, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl) ethane, and 2,6-bis(hydroxyalkyl) cresols, among others.

Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

In some embodiments, additional radiopacity may be desired for the multi-arm polymers. One way to introduce additional radiopacity to the multi-arm polymers is to employ an iodine-containing polyol when forming the multi-arm polymers. Illustrative iodinated polyols include iodine-containing polyols that are known for use as iodinated contrast agents, such as 1,3,5-triiodo-2,4,6-tris-hydroxymethylbenzene, iodixanol, iotrolan, iohexol, ioversol, iopamidol, iohexol impurity J, and iopromide, among others.

In some embodiments of the present disclosure, a hydroxy-terminated multi-arm polymer having multiple polymer arms that comprises a core region and a plurality of polymer arms that each comprise hydrophilic polymer segment and a C1-C6-alkyl end group is used as a precursor polymer to form an amino-alcohol-terminated multi-arm polymer that comprises a core region and a plurality of polymer arms that each comprise an amino-alcohol end group that is attached through an ether group to the hydrophilic polymer segment.

Examples of amino-alcohol groups for used in the present disclosure include for example, C1-C6-alkyl groups that are substituted with an amino group and a hydroxyl group, which include, for example, hydroxyaminomethyl groups, hydroxyaminoethyl groups, hydroxyaminopropyl groups, hydroxyaminobutyl groups, hydroxyaminopentyl groups, or hydroxyaminohexyl groups. These C1-C6-hydroxyaminoalkyl groups are provided on polymer arms that comprise hydrophilic polymer segments having C1-C6-hydroxyaminoalkoxy end groups, including hydroxyaminomethyloxy end groups, hydroxyaminoethyloxy end groups, hydroxyaminopropyloxy end groups, hydroxyaminobutyloxy end groups, hydroxyaminopentyloxy end groups, or hydroxyaminohexyloxy end groups. In some embodiments, the amino-alcohol groups are vicinal amino-alcohol groups in which the amino group and the hydroxyl group are attached to adjacent carbon atoms.

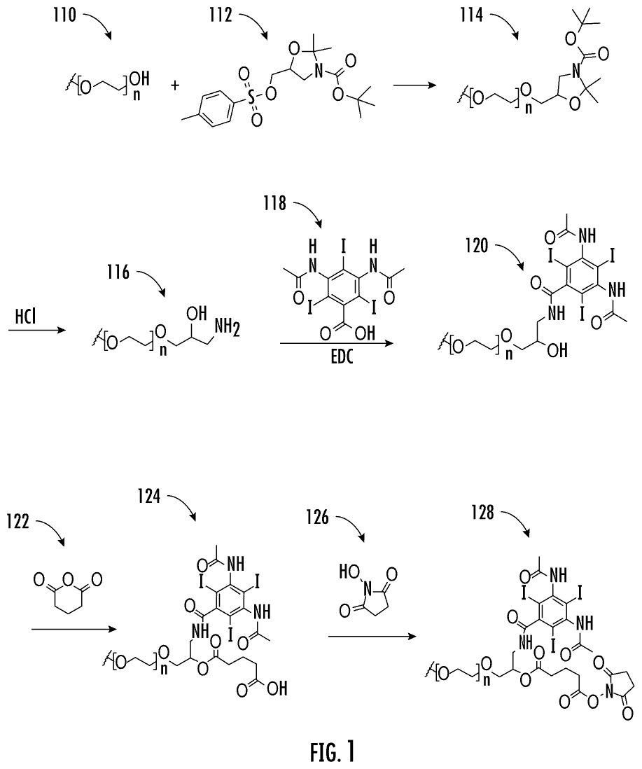

One particular embodiment will now be described with reference to FIG. 1, which employs a hydroxy-terminated multi-arm polymer, specifically, hydroxy-terminated multi-arm polyethylene glycol (PEG) 110, where n represents and integer and may have a value ranging, for example, from 5 to 500. It is noted that only a single arm of the multi-arm polymer is shown, and the remaining arms and the core are not illustrated. In a first step, the hydroxy-terminated multi-arm PEG (110) is reacted with 1,1-dimethylethyl 2,2-dimethyl-5-[[[(4-methylphenyl)sulfonyl]oxy]methyl]-3-oxazolidinecarboxylate (CAS 2288710-60-9) (112) in a substitution reaction [to form a multi-arm polymer (114) in which the terminal hydroxyl group is replaced with a tert-butyl 2,2-dimethyloxazolidine-3-carboxylate group. In a subsequent step, the multi-arm polymer (114) is treated with an acid such as hydrochloric acid or TFA in the presence of water to remove the protective groups, thereby providing an amino-alcohol-terminated multi-arm polymer in which an amino-alcohol end group is attached to the PEG segment of each polymer arm through an ether group, specifically, 2-hydroxy-3-amino-n-propoxy-terminated multi-arm PEG (116).

The amino group of the amino-alcohol-terminated multi-arm polymer is subsequently used for the attachment of an iodinated group to the multi-arm polymer, and the hydroxyl group of the amino-alcohol-terminated multi-arm polymer is subsequently used for the attachment of an electrophilic group to the multi-arm polymer. As seen from the discussion below, the iodinated group is generally attached to the multi-arm polymer before attachment of the electrophilic group.

Turning first to the attachment of the iodinated group, in various embodiments, the amino group provided by the amino-alcohol group of the amino-alcohol-terminated multi-arm polymer may be reacted in an amide coupling reaction with a carboxylic-acid group of a carboxylic-acid-containing iodinated compound. The carboxylic-acid-containing iodinated compound may comprise at least one iodinated aromatic group (e.g., a monocyclic or multicyclic aromatic structure that is substituted with one, two, three, four, five, six or more iodine atoms) and a carboxylic acid group. In preferred embodiments, the amino group may be reacted with the carboxylic acid group in the presence of a suitable amide coupling agent. Examples of suitable amide coupling agents include carbodiimide coupling agents, such as N,N′-dicyclohexylcarbodiimide (DCC), 1-ethyl-3-(3-dimethyl propyl) carbodiimide (EDC), and 1,3-diisopropylcarbodiimide (DIC), N-hydroxybenzotriazole (HOBt), BOP reagent, and TBTU (2-(1H-Benzotriazole-1-yl)-1,1,3,3-tetramethylaminium tetrafluoroborate), among others.

Carboxylic-acid-containing iodinated compounds for use in the present disclosure include triiodobenzoic acid

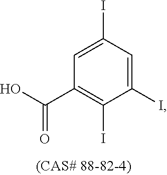 [SMILES: O=C(O)c1cc(I)cc(I)c1I]

diatrizoic acid,

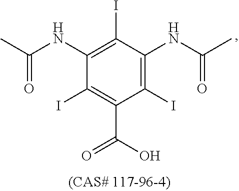 [SMILES: CC(=O)Nc1c(I)c(NC(C)=O)c(I)c(C(=O)O)c1I]

N-acetyl-3,5-diiodo-L-tyrosine,

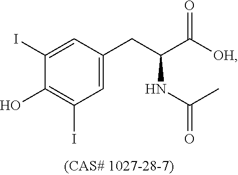 [SMILES: CC(=O)N[C@@H](Cc1cc(I)c(O)c(I)c1)C(=O)O]

N-acetyl-3-diiodo-L-tyrosine,

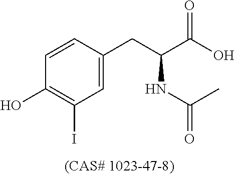 [SMILES: CC(=O)N[C@@H](Cc1ccc(O)c(I)c1)C(=O)O]

and N-acetyl-thyroxine,

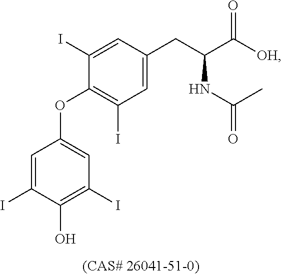 [SMILES: CC(=O)N[C@@H](Cc1cc(I)c(Oc2cc(I)c(O)c(I)c2)c(I)c1)C(=O)O]

among others.

Various additional carboxylic-acid-containing iodinated compounds, along with their CAS numbers, are listed in the following table:

| | | # I |
|---|---|---|
| Name | CAS | atoms |
| 2-Iodobenzoic acid | 88-67-5 | 1 |
| 4-Iodobenzoic acid | 619-58-9 | 1 |
| 3-Iodobenzoic acid | 618-51-9 | 1 |
| 4-Iodopicolinic acid | 405939-79-9 | 1 |
| 3,5-Diiodobenzoic acid | 19094-48-5 | 2 |
| 3,4-Diiodobenzoic acid | 35674-20-5 | 2 |
| 3,5-Diiodo-4-(4-methoxyphenoxy)benzoic acid | 34043-77-1 | 2 |
| Diatrizoate | 117-96-4 | 3 |
| 2,3,5-Triiodobenzoic acid | 88-82-4 | 3 |
| Acetrizoic acid | 85-36-9 | 3 |
| Metrizoate | 1949-45-7 | 3 |
| 3,4,5-Triiodobenzoic acid | 2338-20-7 | 3 |
| Ioxitalamic acid | 28179-44-4 | 3 |
| Iothalamic acid | 2276-90-6 | 3 |
| 2,4,6-Triiodo-1,3,5-benzenetricarboxylic acid | 79211-41-9 | 3 |
| 2,4,6-Triiodobenzoic acid | 2012-31-9 | 3 |
| enzoic acid, 3-[[[2-Hydroxy-1- | 87932-11-4 | 3 |
| (hydroxymethyl)ethyl]amino]carbonyl]-5-[(2- | | |
| hydroxy-1-oxopropyl)amino]-2,4,6- | | |
| triiodo-, (S)-(9CI) | | |
| Ioseric acid | 51876-99-4 | 3 |
| 4-[4-(Acetyloxy)-3-iodophenoxy]-3,5- | 2260-084 | 3 |
| diiodobenzoic acid | | |
| Ioglicic acid | 49755-67-1 | 3 |
| 2,4,6-Triiodo-3-[(1-oxo-3,6,9,12,15- | 16024-67-2 | 3 |
| pentaoxahexadec-1-yl)amino]benzoic acid | | |
| 3-[[(1,1-Dimethylethoxy)carbonyl]amino]- | 2358047-48-8 | 3 |
| 2,4,6-triiodobenzoic acid | | |
| 2,3,4,6-Tetraiodobenzoic acid | 71463-71-3 | 4 |
| Tetraform | 2055-97-2 | 4 |
| 2,3,5,6-Tetraiodo-1,4-benzenedicarboxylic acid | 7606-84-0 | 4 |
| N-Acetyl-O-(4-hydroxy-3,5-diiodophenyl)-3,5- | 26041-51-0 | 4 |
| diiodo-L-tyrosine | | |
| D-Tyrosine, N-[(1,1-dimethylethoxy)carbonyl]- | 89624-64-6 | 4 |
| O-(4-hydroxy-3,5-diiodophenyl)-3,5-diiodo- | | |
| (9CI, ACI) | | |
| 2,3,4,5,6-Pentaiodobenzoic acid | 64385-02-0 | 5 |
| Ioxaglic acid | 59017-64-0 | 6 |
| Ioglycamic acid | 2618-25-9 | 6 |
| Iocarmic acid | 10397-75-8 | 6 |

Carboxylic-acid-containing iodinated compounds for use in the present disclosure can also be formed by reacting an amine-containing iodinated compound with a cyclic anhydride in a ring-opening reaction. The amine-containing iodinated compound may comprise, for example, an iodinated aromatic group and an amine group. The amine-containing iodinated compound can be used to form, for example, a carboxy-C1-C6-alkylamido-containing iodinated compound.

Some specific examples of amine-containing aromatic iodinated compounds are found in the table to follow.

| Name | CAS | # I atoms |
|---|---|---|
| 4-Iodobenzylamine | 39959-59-6 | 1 |
| 5-Amino-N,N′-bis(2,3-dihydroxypropyl)- | 76801-93-9 | 3 |
| 2,4,6-triiodoisophthalamide | | |
| 3,5-Diiodobenzenamine | 35122-96-4 | 2 |
| 2,4,6-Triiodobenzenamine | 24154-37-8 | 3 |

In some embodiments, the amine-containing aromatic iodinated species that is reacted with a cyclic anhydride in a ring-opening reaction is a C₁-C₅-alkyl ester of an iodinated amino acid, preferably a methyl ester of an iodinated amino acid. Specific examples of iodinated amino-acid C₁-C₅-alkyl esters include the following: monoiodo-phenylalanine methyl ester,

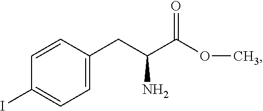 [SMILES: COC(=O)[C@@H](N)Cc1ccc(I)cc1]

monoiodotyrosine methyl ester,

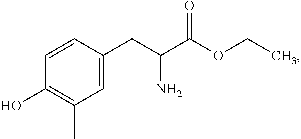 [SMILES: CCOC(=O)C(N)Cc1ccc(O)c(C)c1]

diiodotyrosine methyl ester,

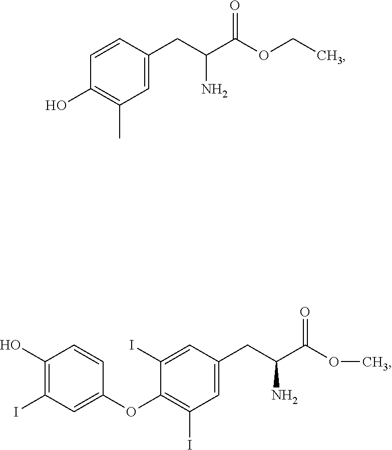 [SMILES: CCOC(=O)C(N)Cc1ccc(O)c(C)c1 ; COC(=O)[C@@H](N)Cc1cc(I)c(Oc2ccc(O)c(I)c2)c(I)c1]

triiodothyronine methyl ester, also known as T3 methyl ester,

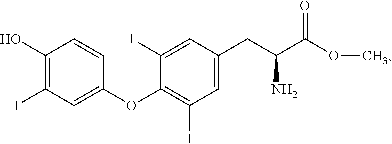 [SMILES: COC(=O)[C@@H](N)Cc1cc(I)c(Oc2ccc(O)c(I)c2)c(I)c1]

tetraiodothyronine methyl ester, also known as thyroxine methyl ester or T4 methyl ester,

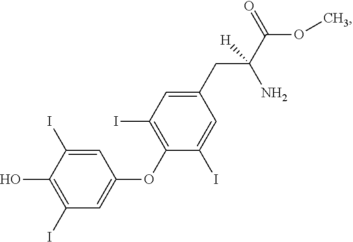 [SMILES: COC(=O)[C@@H](N)Cc1cc(I)c(Oc2cc(I)c(O)c(I)c2)c(I)c1]

thyroxine methyl ester,

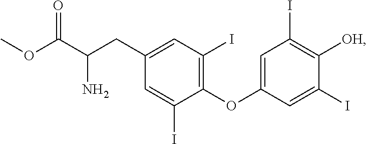 [SMILES: COC(=O)C(N)Cc1cc(I)c(Oc2cc(I)c(O)c(I)c2)c(I)c1]

and bis-thyroxine methyl ester (w/multi-arm-linker),

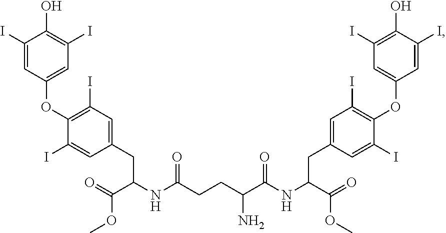 [SMILES: COC(=O)C(Cc1cc(I)c(Oc2cc(I)c(O)c(I)c2)c(I)c1)NC(=O)CCC(N)C(=O)NC(Cc1cc(I)c(Oc2cc(I)c(O)c(I)c2)c(I)c1)C(=O)OC]

and 6-iodo-L-DOPA methyl ester, among others.

Various anhydrides are listed below.

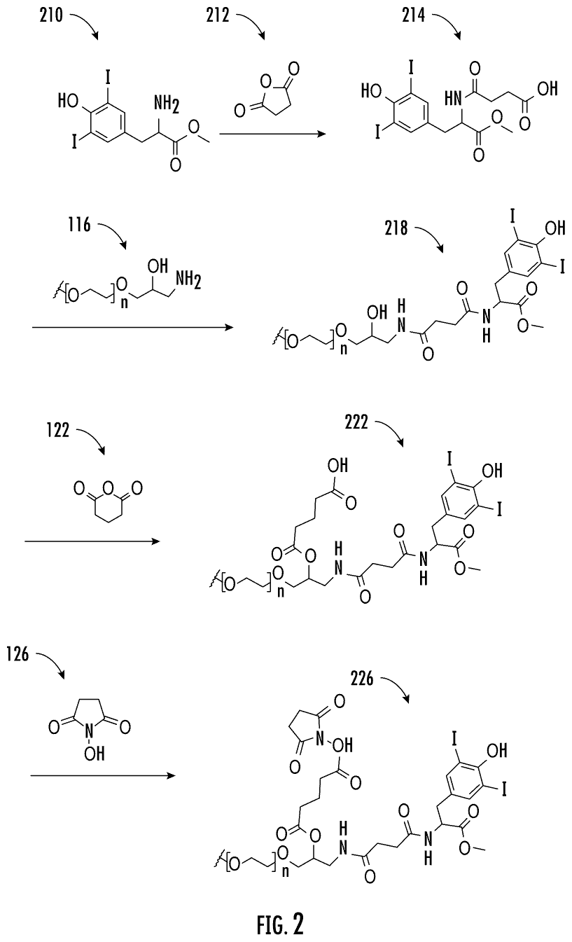

In a specific embodiment shown in FIG. 2, an iodinated amino-acid C₁-C₅-alkyl ester, specifically, diiodotyrosine methyl ester (210), is reacted in a ring-opening reaction with a cyclic anhydride, specifically, succinic anhydride (212), to form a carboxyalkylamido-modified iodinated amino-acid C₁-C₅-alkyl ester, specifically, 4-[[1-[(4-hydroxy-3,5-diiodo-phenyl)methyl]-2-methoxy-2-oxo-ethyl]amino]-4-oxo-butanoic acid (214).

In various embodiments, the amino groups provided by an amino-alcohol-terminated multi-arm polymer, which comprises a core region and a plurality of polymer arms that each comprise an amino-alcohol end group that is attached through an ether group to a hydrophilic polymer segment, are reacted with a carboxylic-acid group of a carboxylic-acid-containing iodinated compound in the presence of a coupling agent to form a multi-arm polymer which comprises a core region and a plurality of polymer arms that each comprise a residue of the carboxylic-acid-containing iodinated molecule, which is attached to a residue of each amino-alcohol group of the amino-alcohol-terminated multi-arm polymer through a linkage comprising an amide group, which amino-alcohol end group residue is in turn attached to the hydrophilic polymer segment through an ether group.

In a specific embodiment shown in FIG. 1, the amino groups of the 2-hydroxy-3-amino-n-propoxy-terminated multi-arm PEG (116) of FIG. 1 are reacted in an amide coupling reaction with diatrizoic acid (118) in the presence of EDC as a coupling agent, to form 2-hydroxy-3-(3,5-diacetamido-2,4,6-triiodobenzamido)-n-propoxy-terminated multi-arm PEG (120).

In another specific embodiment shown in FIG. 2, the amino groups of the 2-hydroxy-3-amino-n-propoxy-terminated multi-arm PEG (116) of FIG. 1 are reacted in an amide coupling reaction with a carboxyalkylamido-modified iodinated amino-acid C₁-C₅-alkyl ester, specifically, 4-[[1-[(4-hydroxy-3,5-diiodo-phenyl)methyl]-2-methoxy-2-oxo-ethyl]amino]-4-oxo-butanoic acid (214), preferably in the presence of a coupling agent, to form 2-hydroxy-3-(4-[[1-[(4-hydroxy-3,5-diiodo-phenyl)methyl]-2-methoxy-2-oxo-ethyl]amino]-4-oxo-butanoic acid)-n-propoxy-terminated multi-arm PEG (218). It is noted that the multi-arm PEG (218) contains an iodinated amino-acid C₁-C₅-alkyl ester residue, specifically, a diiodotyrosine methyl ester residue, which is attached to an amino-alcohol residue through a linkage that contains two amide groups.

Turning now to the attachment of the electrophilic group, in various embodiments, the hydroxyl group provided by the amino-alcohol group of the amino-alcohol-terminated multi-arm polymer may be reacted in a ring-opening reaction with a cyclic anhydride to form a carboxylic acid group that is linked to a residue of the amino-alcohol group of the amino-alcohol-terminated multi-arm polymer through an ester-containing linkage.

Cyclic anhydrides for use in the present disclosure include, for example, various cyclic anhydrides having a ring that comprises a —C₂-C₈-alkylene-group, including, for example, succinic anhydride,

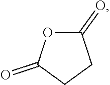

which comprises a dimethylene group, glutaric anhydride,

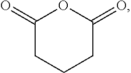

which comprises a trimethylene group, adipic anhydride,

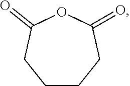

which comprises a tetramethylene group, pimelic anhydride,

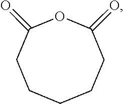

which comprises a pentamethylene group, and suberic anhydride,

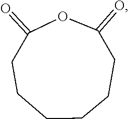 [SMILES: O=C1CCCCCCC(=O)O1]

which comprises a hexamethylene group, among others.

These and other specific examples of anhydrides are listed in the following table.

| Species | CAS # |
|---|---|
| Succinic anhydride | 108-30-5 |
| Glutaric anhydride | 108-55-4 |
| Adipic anhydride | 2035-75-8 |
| Pimelic anhydride | 10521-07-0 |
| Malonic anhydride | 15159-48-5 |
| Itaconic anhydride | 2170-03-8 |
| Maleic anhydride | 108-31-6 |
| 3-Oxabicyclo[3.1.0]hexane-2,4-dione | 5617-74-3 |
| 3-Oxabicyclo[3.2.0]heptane-2,4-dione | 4462-96-8 |
| 3-Methylglutaric anhydride | 4166-53-4 |
| Methylsuccinic anhydride | 4100-80-5 |
| 2,2-Dimethylsuccinic anhydride | 17347-61-4 |
| 6,6-Dimethyl-3-oxabicyclo[3.1.0]hexane-2,4-dione | 67911-21-1 |
| Dihydro-4,4-dimethyl-2H-pyran-2,6(3H)-dione | 4160-82-1 |
| Diglycolic anhydride | 4480-83-5 |
| Oxane-2,4,6-trione | 10521-08-1 |
| Dihydro-4-(2-methylpropyl)-2H-pyran-2,6(3H)-dione | 185815-59-2 |

For instance, when a cyclic anhydride having a ring that comprises a —C₂-C₈-alkylene-group is reacted in a ring-opening reaction with a hydroxyl group, the result is a carboxyl-C₂-C₈-alkylene-carbonyloxy-group, for example, a carboxydimethylenecarbonyloxy- group in the case of succinic anhydride, a carboxytrimethylenecarbonyloxy- group in the case of glutaric anhydride, a carboxytetramethylenecarbonyloxy- group in the case of adipic anhydride, a carboxypentamethylenecarbonyloxy- group in the case of pimelic anhydride, a carboxyhexamethylenecarbonyloxy- group in the case of suberic anhydride, and so forth.

A reactive moiety, for example, an electrophilic moiety, may then be linked to through the carboxyl group of the cyclic anhydride residue that arises from the ring-opening reaction.

In some embodiments, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic acid group of the cyclic anhydride residue in the presence of a suitable coupling agent (e.g., a carbodiimide coupling agent such as one of those described above) to form a reactive cyclic imide ester group (e.g., a succinimide ester group, a maleimide ester group, a glutarimide ester group, a phthalimide ester group, a diglycolimide ester group, a bicyclo[2.2.1]hept-5-ene-2,3-dicarboxylic acid imide ester group, etc.) that is linked to the amino-alcohol group residue, more particularly, a reactive cyclic imide ester group that is linked to the residue of the amino-alcohol group through an ester-containing linkage, which was formed in the cyclic-anhydride ring-opening reaction described above. Examples include cyclic-imidyl-oxycarbonyl-C₂-C₈-alkylene-carbonyloxy-groups, for example, a cyclic-imidyl-carboxydimethylenecarbonyloxy-group (also referred to as a cyclic-imidyl-succinate group) in the case of succinic anhydride, a cyclic-imidyl-carboxytrimethylenecarbonyloxy-group (also referred to as a cyclic-imidyl-glutarate group) in the case of glutaric anhydride, a cyclic-imidyl-carboxytetramethylenecarbonyloxy-group (also referred to as a cyclic-imidyl-adipate group) in the case of adipic anhydride, a cyclic-imidyl-carboxypentamethylenecarbonyloxy-group (also referred to as a cyclic-imidyl-pimelate group) in the case of pimelic anhydride, a cyclic-imidyl-carboxyhexamethylenecarbonyloxy-group (also referred to as a cyclic-imidyl-suberate group) in the case of suberic anhydride, and so forth. In this way, a variety of activated ester groups can be formed.

For example, in the particular case of N-hydroxysuccinimide as an N-hydroxy cyclic imide compound and malonic acid anhydride, glutaric acid anhydride, succinic acid anhydride, adipic acid anhydride, or diglycolic acid anhydride, respectively, as a cyclic anhydride, exemplary reactive groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound and malonic acid anhydride, glutaric acid anhydride, succinic acid anhydride, adipic acid anhydride, or diglycolic acid anhydride, respectively, as a cyclic anhydride, exemplary reactive groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound and malonic acid anhydride, glutaric acid anhydride, succinic acid anhydride, adipic acid anhydride, or diglycolic acid anhydride, respectively, as a cyclic anhydride, exemplary reactive groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound and malonic acid anhydride, glutaric acid anhydride, succinic acid anhydride, adipic acid anhydride, or diglycolic acid anhydride, respectively, as a cyclic anhydride, exemplary reactive groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound and malonic acid anhydride, glutaric acid anhydride, succinic acid anhydride, adipic acid anhydride, or diglycolic acid anhydride, respectively, as a cyclic anhydride, exemplary reactive groups include phthalimidyl malonate groups, phthalimidyl glutarate groups, phthalimidyl succinate groups, phthalimidyl adipate groups, and phthalimidyl diglycolate groups, among others.

Thus, in various embodiments, the hydroxyl groups provided by amino-alcohol groups of an amino-alcohol-terminated multi-arm polymer, are reacted with a cyclic anhydride to form a multi-arm polymer which comprises a plurality of polymer arms that each comprise a carboxylic acid group that is linked through an ester-containing linkage to a residue of the amino-alcohol group of the amino-alcohol-terminated multi-arm polymer. Then, the carboxylic acid groups provided by the ring-opening reaction of the cyclic anhydride are reacted in an amide coupling reaction with an N-hydroxy cyclic imide compound to form a cyclic-imidyl ester groups that are coupled through a hydrolysable ester-containing linkages to residues of the amino-alcohol group.

In a particular embodiment shown in FIG. 1, the hydroxy groups of the 2-hydroxy-3-(3,5-diacetamido-2,4,6-triiodobenzamido)-n-propoxy-terminated multi-arm PEG (120) are reacted in a ring-opening reaction with glutaric anhydride (122) to form 2-(glutaric acid)-3-(3,5-diacetamido-2,4,6-triiodobenzamido)-n-propoxy-terminated multi-arm PEG (124), also referred to herein as 2-(carboxytrimethylenecarbonyloxy)-3-(3,5-diacetamido-2,4,6-triiodobenzamido)-n-propoxy-terminated multi-arm PEG, which is then reacted in an amide coupling reaction with N-hydroxy succinimide (126) (typically in the presence of a coupling agent) to form 2-(succinimidyl glutarate)-3-(3,5-diacetamido-2,4,6-triiodobenzamido)-n-propoxy-terminated multi-arm PEG (128) also referred to as 2-(succinimidyloxycarbonyltrimethylenecarbonyloxy)-3-(3,5-diacetamido-2,4,6-triiodobenzamido)-n-propoxy-terminated multi-arm PEG.

In another particular embodiment shown in FIG. 2, the hydroxy groups of the 2-hydroxy-3-(4-[[1-[(4-hydroxy-3,5-diiodo-phenyl)methyl]-2-methoxy-2-oxo-ethyl]amino]-4-oxo-butanoic acid)-n-propoxy-terminated multi-arm PEG (218) are reacted in a ring-opening reaction with glutaric anhydride (122) to form 2-(glutaric acid)-3-(4-[[1-[(4-hydroxy-3,5-diiodo-phenyl)methyl]-2-methoxy-2-oxo-ethyl]amino]-4-oxo-butanoic acid)-n-propoxy-terminated multi-arm PEG (222), which is then reacted in an amide coupling reaction with N-hydroxy succinimide (126) (typically in the presence of a coupling agent) to form 2-(succinimidyl glutarate)-3-(4-[1-[(4-hydroxy-3,5-diiodo-phenyl)methyl]-2-methoxy-2-oxo-ethyl]amino]-4-oxo-butanoic acid)-n-propoxy-terminated multi-arm PEG (226).

In other embodiments, radiopaque reactive polymers for use in the present disclosure include reactive multi-arm polymers that comprise a core region, a plurality of polymer arms linked to the core region, at least three of the polymer arms each comprising a hydrophilic polymer segment and residue of a halide-substituted cyclic anhydride, specifically a bromide-substituted cyclic anhydride or an iodide-substituted cyclic anhydride, that is attached to the hydrophilic polymer segment through a hydrolysable ester group, an electrophilic group attached to each bromine-substituted cyclic anhydride residue, and a residue of a hydroxyl-containing iodinated molecule attached to each bromine-substituted cyclic anhydride residue through a linkage comprising an ether group and, optionally, an amide group.

Various examples of core regions, hydrophilic polymer segments, and electrophilic groups are described above.

In some embodiments of the present disclosure, a hydroxyalkyl-terminated multi-arm polymer having multiple polymer arms that comprises a core region and a plurality of polymer arms that each comprise a hydroxyalkyl-terminated hydrophilic polymer segment (e.g., a C1-C6-hydroxyalkyl-terminated hydrophilic polymer segment) is used as a precursor polymer to form a carboxyhaloalkyl-terminated hydrophilic polymer segment in which a carboxyhaloalkyl group is linked to a residue of the hydroxyalkyl-terminated multi-arm polymer through an ester-containing linkage. In some of these embodiments, the hydroxyalkyl-terminated multi-arm polymer is reacted in a ring opening reaction with a halogen-substituted cyclic anhydride compound to form the carboxyhaloalkyl group that is linked to a residue of the hydroxyalkyl-terminated multi-arm polymer through an ester-containing linkage.

Examples of such carboxyhaloalkyl-terminated hydrophilic polymer segments include carboxyhaloalkylcarbonyloxy-terminated hydrophilic polymer segments, for example, carboxy-monohalo-C1-C6-alkyl-carbonyloxy-terminated hydrophilic polymer segments, carboxy-dihalo-C1-C6-alkyl-carbonyloxy-terminated hydrophilic polymer segments, carboxy-trihalo-C1-C6-alkyl-carbonyloxy-terminated hydrophilic polymer segments, and carboxy-tetrahalo-C1-C6-alkyl-carbonyloxy-terminated hydrophilic polymer segments, among others, where the halo groups can be bromo groups or iodo groups.

Examples of halogen-substituted cyclic anhydride compounds include monohalogen-substituted-cyclic anhydride compounds, dihalogen-substituted-cyclic anhydride compounds, trihalogen-substituted-cyclic anhydride compounds, and tetrahalogen-substituted-cyclic anhydride compounds, among others. Examples of halogen-substituted cyclic anhydride compounds include halogen-substituted versions of the cyclic anhydride compounds described above. Particular examples include, for example, monohalogen-substituted-succinic anhydride, dihalogen-substituted-succinic anhydride, trihalogen-substituted-succinic anhydride, tetrahalogen-substituted-succinic anhydride, monohalogen-substituted-glutaric anhydride, dihalogen-substituted-glutaric anhydride, trihalogen-substituted-glutaric anhydride, tetrahalogen-substituted-glutaric anhydride, monohalogen-substituted-adipic anhydride, dihalogen-substituted-adipic anhydride, trihalogen-substituted-adipic anhydride, tetrahalogen-substituted-adipic anhydride, monohalogen-substituted-pimelic anhydride, dihalogen-substituted-pimelic anhydride, trihalogen-substituted-pimelic anhydride, tetrahalogen-substituted-pimelic anhydride, monohalogen-substituted-suberic anhydride, dihalogen-substituted-suberic anhydride, trihalogen-substituted-suberic anhydride, and tetrahalogen-substituted-suberic anhydride, among many others, where the halogen groups can be bromine groups or iodine groups.

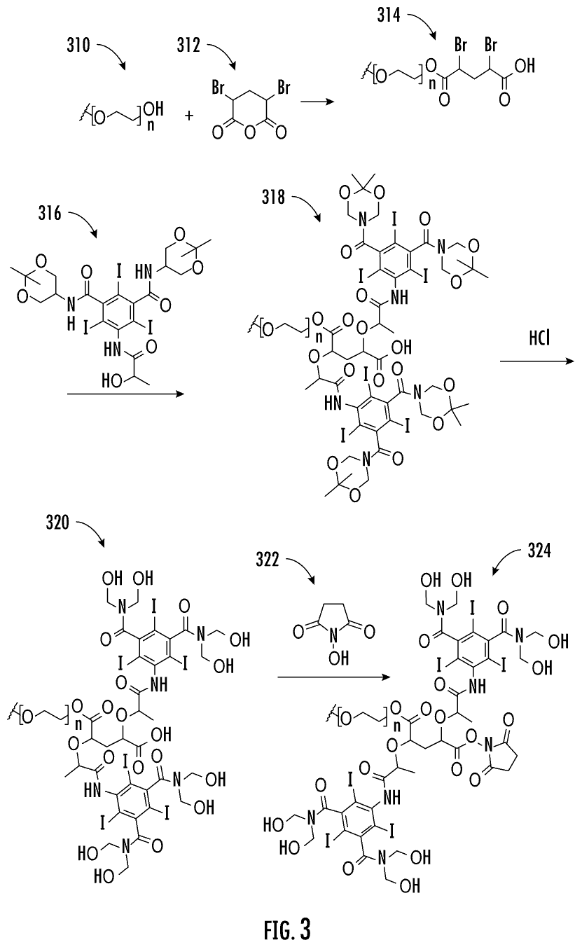

One particular embodiment will now be described with reference to FIG. 3, which employs a hydroxyalkyl-terminated multi-arm polymer, specifically, hydroxyethyl-terminated multi-arm polyethylene glycol (PEG) 310, where n represents and integer and may have a value ranging, for example, from 5 to 500 (only a single arm of the multi-arm polymer is shown, and the remaining arms and the core are not illustrated). In a first step, the hydroxyethyl-terminated multi-arm PEG (310) is reacted with dibromoglutaric anhydride (312) in a ring-opening reaction to form a multi-arm polymer (314) in which the terminal hydroxyl group of each polymer arm is replaced with a carboxybromoalkylcarbonyloxy group, specifically, a carboxy-2,4-dibromopentanedioic acid group.

In some embodiments, once the carboxybromoalkyl end groups are formed, a hydroxyalkyl group of a hydroxyalkyl-containing iodinated aromatic compound having one or more hydroxyalkyl substituents (e.g., one or more primary or secondary alcohol substituents) is reacted with a base such as potassium carbonate (K₂CO₃) or sodium hydride (NaH) to form an alkoxide ion, which acts as a nucleophile that displaces one or more bromine groups from the bromoalkyl-containing end groups to yield one or more ether linkages

Specific examples of hydroxyalkyl-containing iodinated aromatic compounds include those that comprise one or more monocyclic or multicyclic aromatic groups, substituted with (a) one or more iodine groups (e.g., one two, three, four, five, six, seven, eight, nine, ten or more iodine atoms) and (b) one or more hydroxyl-containing groups independently selected from one or more hydroxyl groups and/or one or more C1-C4-hydroxyalkyl groups (e.g., C1-C4-monohydroxyalkyl groups, C1-C4-dihydroxyalkyl groups, C1-C4-trihydroxyalkyl groups, C1-C4-tetrahydroxyalkyl groups, etc.), among others, which C1-C4-hydroxyalkyl groups may be linked to the one or more monocyclic or multicyclic aromatic groups directly or through any suitable linking moiety, which may be selected, for example, from alkyl groups (e.g., alkyl groups containing one carbon, two carbons, three carbons, four carbons, etc.), amide groups, ether groups, urea groups, urethane groups, and combinations thereof, among others.

More specific examples of hydroxyalkyl-containing iodinated aromatic compounds include those presented in the table below.

| | | # of OH |
|---|---|---|
| Compound | CAS# | Groups |
| Iopromide | 73334-07-3 | 4 |
| 5-Acetamido-N,N′-bis(2,3-dihydroxypropyl)- | 31127-80-7 | 4 |
| 2,4,6-triiodoisophthalamide | | |
| Metrizamide | 31112-62-6 | 4 |
| 1,3-Benzenedicarboxamide, 5-(acetylamino)- | 76350-28-2 | 4 |
| N1,N3-bis(2,3-dihydroxypropyl)-2,4,6- | | |
| triiodo-N1-methyl- (ACI) | | |
| Iopamidol | 60166-93-0 | 5 |
| Iomeprol | 78649-41-9 | 5 |
| 1,3-Benzenedicarboxamide, N1,N3-bis[2- | 60208-45-9 | 5 |
| hydroxy-1-(hydroxymethyl)ethyl]-5-[(2-hydroxy- | | |
| 1-oxopropyl)amino]-2,4,6-triiodo- (ACI) | | |
| Ioxilan | 107793-72-6 | 5 |
| Iopentol | 89797-00-2 | 5 |
| Ioversol | 87771-40-2 | 6 |
| Iohexol | 66108-95-0 | 6 |
| Iobitridol | 136949-58-1 | 6 |
| Iodixanol | 92339-11-2 | 9 |
| 1,3-Benzenedicarboxamide, 5,5′-[(1,3-dioxo-1,3- | 79770-24-4 | 12 |
| propanediyl)bis(methylimino)]bis[N,N′-bis[2,3- | | |
| dihydroxy-1-(hydroxymethyl)propyl]-2,4,6- | | |
| triiodo- (9CI, ACI) | | |

In a particular example, and with reference again to FIG. 3, a hydroxyalkyl-containing iodinated compound, specifically, acetal-protected iopamidol (316), in which two vicinal-diol hydroxyl groups are protected and a non-vicinal-diol hydroxyl group remains unprotected, is reacted with a multi-arm PEG having end groups that correspond to bromine-substituted cyclic anhydride compound residues, specifically, carboxy-2,4-dibromopentanedioic acid-terminated multi-arm PEG (314) having end groups that correspond to dibromoglutaric acid residues, in the presence of a base such as potassium carbonate to form an ether linkage between a residue of the hydroxyalkyl-containing iodinated compound and a residue of the bromine-substituted cyclic anhydride compound at a position previously occupied by each of the bromine groups. In this way, a multi-arm PEG (318) is formed in which a bromine-substituted cyclic anhydride compound residue, specifically, a dibromoglutaric anhydride residue, is attached to each PEG segment through a hydrolysable ester group and one or more hydroxyl-containing iodinated compound residues, specifically, two acetal-protected iopamidol residues, are attached to the bromine-substituted cyclic anhydride compound residue through an ether group.

In a subsequent step, the multi-arm PEG (318) treated with an acid such as hydrochloric acid or TFA to remove the acetal protective groups, thereby providing an iodinated multi-arm PEG (320) in which a bromine-substituted cyclic anhydride compound residue, specifically, a dibromoglutaric anhydride residue, is attached to each PEG segment through a hydrolysable ester group and one or more hydroxyl-containing iodinated compound residues, specifically, two iopamidol residues, are attached to the cyclic anhydride compound residue through an ether group.

It can be seen that the bromine-substituted cyclic anhydride compound residue contains a carboxyalkyl group, which may be further used to provide a multi-arm polymer with an electrophilic group. In some embodiments, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic acid group of the bromine-substituted cyclic anhydride compound residue in the presence of a suitable coupling agent (e.g., a carbodiimide coupling agent such as one of those described above) to form a reactive cyclic imide ester group (e.g., a succinimide ester group, a maleimide ester group, a glutarimide ester group, a phthalimide ester group, a diglycolimide ester group, a bicyclo[2.2.1]hept-5-ene-2,3-dicarboxylic acid imide ester group, etc.) that is linked to the bromine-substituted cyclic anhydride compound residue.

In the particular embodiment shown in FIG. 3, the iodinated multi-arm PEG (320) is reacted in an amide coupling reaction with N-hydroxy succinimide (322), typically in the presence of a coupling agent, to form a reactive iodinated multi-arm PEG (324) in which the polymer arms each comprise a hydrophilic polymer segment, specifically, a PEG segment, and a residue of a bromine-substituted cyclic anhydride compound, specifically, a dibromoglutaric anhydride residue, that is attached to the PEG segment through a hydrolysable ester group, wherein a cyclic-imidyl group, specifically, a succinimidyl group, is attached to the dibromoglutaric anhydride residue through an ester group, and residues of two hydroxyl-containing iodinated compounds, specifically, iopamidol residues, are each attached to the dibromoglutaric anhydride residue through a linkage comprising an ether group.

In other embodiments, radiopaque reactive polymers for use in the present disclosure include reactive multi-arm polymers that comprise a core region, a plurality of polymer arms linked to the core region, at least three of the polymer arms each comprising a hydrophilic polymer segment and residue of a carboxylic-acid-substituted cyclic anhydride that is attached to the hydrophilic polymer segment through a hydrolysable ester group, an electrophilic group attached to each carboxylic-acid-substituted cyclic anhydride residue, and a residue of a hydroxyl-containing iodinated molecule, which comprises an iodinated aromatic group and a hydroxyl group, attached to each carboxylic-acid-substituted cyclic anhydride residue through a linkage that comprises a urethane group and an amide group.

Various examples of core regions, hydrophilic polymer segments, and electrophilic groups are described above.

In some embodiments of the present disclosure, a hydroxyalkyl-terminated multi-arm polymer having multiple polymer arms that comprises a core region and a plurality of polymer arms that each comprise a hydroxyalkyl-terminated hydrophilic polymer segment (e.g., a C1-C6-hydroxyalkyl-terminated hydrophilic polymer segment) is reacted in a ring opening reaction with a carboxyl-protected carboxylic-acid-substituted cyclic anhydride compound to form a multi-arm polymer having multiple polymer arms that comprises a core region and a plurality of polymer arms that each comprise a carboxylic-acid-terminated hydrophilic polymer segment in which an end group that contains a protected carboxyl group and an unprotected carboxyl group is linked to a residue of the alcohol-terminated multi-arm polymer through an ester-containing linkage. In some embodiments, the end group is a C1-C6-alkyl group that is substituted with an unprotected carboxyl group and one or more protected carboxyl groups. Examples of carboxylic acid protecting groups include esters such as methyl esters, benzyl esters, tert-butyl esters, allyl esters, and trimethylsilylethyl esters.

Examples of carboxylic-acid-substituted cyclic anhydride compounds include mono-carboxylic-acid-substituted-cyclic anhydride compounds, di-carboxylic-acid-substituted-cyclic anhydride compounds, tri-carboxylic-acid-substituted-cyclic anhydride compounds, and tetra-carboxylic-acid-substituted-cyclic anhydride compounds, among others. Examples of carboxylic-acid-substituted cyclic anhydride compounds include-carboxylic-acid-substituted versions of the cyclic anhydride compounds described above. Particular examples include, for example, mono-carboxylic-acid-substituted-succinic anhydride, di-carboxylic-acid-substituted-succinic anhydride, tri-carboxylic-acid-substituted-succinic anhydride, tetra-carboxylic-acid-substituted-succinic anhydride, mono-carboxylic-acid-substituted-glutaric anhydride, di-carboxylic-acid-substituted-glutaric anhydride, tri-carboxylic-acid-substituted-glutaric anhydride, tetra-carboxylic-acid-substituted-glutaric anhydride, mono-carboxylic-acid-substituted-adipic anhydride, di-carboxylic-acid-substituted-adipic anhydride, tri-carboxylic-acid-substituted-adipic anhydride, tetra-carboxylic-acid-substituted-adipic anhydride, mono-carboxylic-acid-substituted-pimelic anhydride, di-carboxylic-acid-substituted-pimelic anhydride, tri-carboxylic-acid-substituted-pimelic anhydride, tetra-carboxylic-acid-substituted-pimelic anhydride, mono-carboxylic-acid-substituted-suberic anhydride, di-carboxylic-acid-substituted-suberic anhydride, tri-carboxylic-acid-substituted-suberic anhydride, and tetra-carboxylic-acid-substituted-suberic anhydride, among many others.

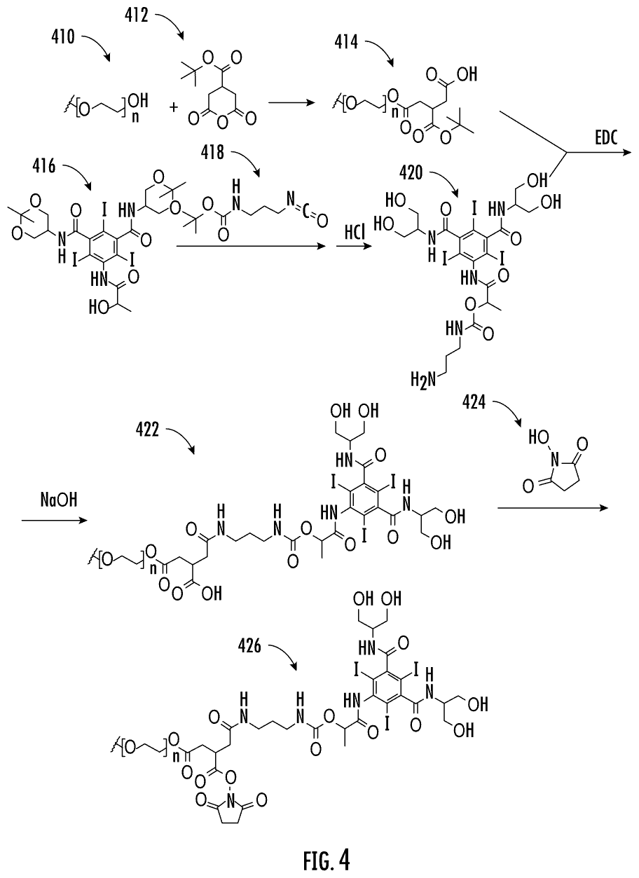

One particular embodiment is illustrated in FIG. 4, which employs a hydroxyalkyl-terminated multi-arm polymer, specifically, hydroxyethyl-terminated multi-arm polyethylene glycol (PEG) 410, where n represents and integer and may have a value ranging, for example, from 5 to 500 (only a single arm of the multi-arm polymer is shown, and the remaining arms and the core are not illustrated). In a first step, the hydroxyethyl-terminated multi-arm PEG (410) is reacted with 1,1-Dimethylethyl tetrahydro-2,6-dioxo-2H-pyran-4-carboxylate (412) in a ring-opening reaction to form a multi-arm polymer (414) in which the terminal hydroxyl group is replaced with a 3-tert-butoxycarbonyl-5-methoxy-5-oxo-pentanoic acid group, specifically, a carboxy-trimethylene-carbonyloxy group in which the trimethylene group is substituted with a tert-butyl-protected-carboxy group.

Once a multi-arm polymer having multiple polymer arms that comprises a core region and a plurality of polymer arms that each comprise a carboxylic-acid-terminated hydrophilic polymer segment in which an end group that contains a protected carboxyl group and an unprotected carboxyl group is formed, the unprotected carboxylic-acid group is then available for further reaction in an amide coupling reaction with an amine-containing aromatic iodinated compound. In preferred embodiments, the amino group may be reacted with the carboxylic acid group in the presence of a suitable coupling agent, such as those described above.

Various amine-containing aromatic iodinated compounds are described above.

In some embodiments, the amine-containing aromatic iodinated compound is formed from a hydroxyalkyl-containing iodinated aromatic compound such as those described above, among others. The hydroxyalkyl-containing iodinated aromatic compound may comprise, for example, at least one iodinated aromatic group (e.g., a monocyclic or multicyclic aromatic structure that is substituted with one, two, three, four, five, six or more iodine atoms) and at least one hydroxyl group. In some embodiments, a hydroxyalkyl-containing iodinated aromatic compound is reacted with an isocyanate group of an amine-protected, isocyanate-containing amine compound, followed by deprotection of the amine group to form an amine-containing iodinated aromatic compound in which an amine group is coupled to an iodinated aromatic group through a urea-group-containing linkage. Examples of amine-protective groups for use in the present disclosure include tert-butoxycarbonyl (boc) groups, carboxybenzyl (CBz) or (Z) groups, trifluoroacetyl (TFA) groups, and 9-fluorenylmethoxycarbonyl (Fmoc) groups, among others.

Turning again to FIG. 4, a hydroxyalkyl-containing iodinated aromatic compound, specifically, acetal-protected iopamidol (416), in which two vicinal-diol hydroxyl groups are protected and a non-vicinal-diol hydroxyl group remains unprotected, is reacted with an isocyanate group of an amine-protected, isocyanate-containing amine compound, specifically, 1,1-Dimethylethyl N-(3-isocyanatopropyl) carbamate (418) followed by deprotection of the acetal protection and the boc protection using an acid such as HCl to form an amine-containing iodinated aromatic compound in which an amine group is coupled to an iodinated aromatic group through a urea-group-containing linkage, specifically, an amine-containing iodinated aromatic compound (420) in which an aminopropyl group is coupled to an ipamidol residue through a urea linkage.

Regardless of how the amine-containing iodinated aromatic compound is obtained, the amine-containing iodinated aromatic compound may be reacted in an amide coupling process with a multi-arm polymer having multiple polymer arms that comprises a core region and a plurality of polymer arms that each comprise a carboxylic-acid-terminated hydrophilic polymer segment in which an end group contains a protected carboxyl group and an unprotected carboxyl group as described above. Preferably the amide coupling is performed in the presence of a suitable coupling agent (e.g., a carbodiimide coupling agent, such as one of those described above). After the amide coupling is complete, the protection is removed from the protected carboxyl group, thereby providing an unprotected carboxyl group which is available for further reactions.

In a particular embodiment show in FIG. 4, the amino group of the previously described amine-containing iodinated aromatic compound (420), in which an aminopropyl group is coupled to an ipamidol residue through a urea linkage, is reacted in an amide coupling process with the unprotected carboxyl group of the previously described a multi-arm polymer (414), which contains a protected-carboxy-substituted carboxyalkylcarbonyloxy group, specifically, a carboxy-trimethylene-carbonyloxy group in which the trimethylene group is substituted with a tert-butyl-protected-carboxy group. The amide coupling is performed in the presence of EDC as a coupling agent. The tert-butyl ester protection is then removed, for example, in the presence of a base such as NaOH, thereby providing a multi-arm PEG (422) in which a carboxylic-acid-substituted cyclic anhydride residue, specifically, a 3-carboxy-glutaric anhydride residue, is attached to each PEG segment through a hydrolysable ester group and one or more hydroxyl-containing iodinated compound residues, specifically, an iopamidol residue, is attached to the carboxylic-acid-substituted cyclic anhydride residue through a urethane group and an amide group.

It can be seen that the carboxylic-acid-substituted cyclic anhydride compound residue contains a carboxyalkyl group, which may be further used to provide a multi-arm polymer with an electrophilic group. In some embodiments, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic acid group of the carboxylic-acid-substituted cyclic anhydride compound residue in the presence of a suitable coupling agent (e.g., a carbodiimide coupling agent such as one of those described above) to form a reactive cyclic imide ester group (e.g., a succinimide ester group, a maleimide ester group, a glutarimide ester group, a phthalimide ester group, a diglycolimide ester group, a bicyclo[2.2.1]hept-5-ene-2,3-dicarboxylic acid imide ester group, etc.) that is linked to the carboxylic-acid-substituted cyclic anhydride compound residue.

In the particular embodiment shown in FIG. 4, the multi-arm PEG (422) is reacted in an amide coupling reaction with N-hydroxy succinimide (424), in the presence of a coupling agent such as DCC, to form a multi-arm PEG (426) in which the polymer arms each comprise a hydrophilic polymer segment, specifically, a PEG segment, and a residue of a carboxylic-acid-substituted cyclic anhydride compound, specifically, a 3-carboxy-glutaric anhydride residue, that is attached to the PEG segment through a hydrolysable ester group, wherein a cyclic-imidyl group, specifically, a succinimidyl group, is attached to the 3-carboxy-glutaric anhydride residue through an ester group, and a residue of a hydroxyl-containing iodinated compound, specifically, an iopamidol residue, is attached to the 3-carboxy-glutaric anhydride residue through a urethane group and an amide group.

In some aspects, the present disclosure provides radiopaque hydrolysable crosslinked hydrogels that comprise crosslinked reaction product of (a) a polyamino compound and (b) a radiopaque reactive polymer that comprises electrophilic groups, such as any of the radiopaque reactive polymers described above, which are reactive with amino groups of the polyamino compound.

Polyamino compounds suitable for use in the present disclosure include, for example, polyamines that contain at least two amino (—NH₂) groups (e.g., 2, 3, 4, 5, 6, 7, 8, 9, 10 or more amino groups in some embodiments). Polyamino compounds suitable for use in the present disclosure include those that comprise a plurality of —(CH₂)ₓ—NH₂ groups where x is 0, 1, 2, 3, 4, 5 or 6. Polyamino compounds suitable for use in the present disclosure include polyamino compounds that comprise basic amino-acid residues, including residues of amino-acids having two or more primary amine groups, such as lysine and ornithine, for example, polyamines that comprise from 2 to 10 lysine and/or ornithine amino-acid residues (e.g., dilysine, trilysine, tetralysine, pentalysine, diornithine, triornithine, tetraornithine, pentaornithine, etc.).

Further particular examples of polyamino compounds which may be used as the multifunctional compound include ethylenetriamine, diethylene triamine, hexamethylenetriiamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, and poly(allyl amine), among others.

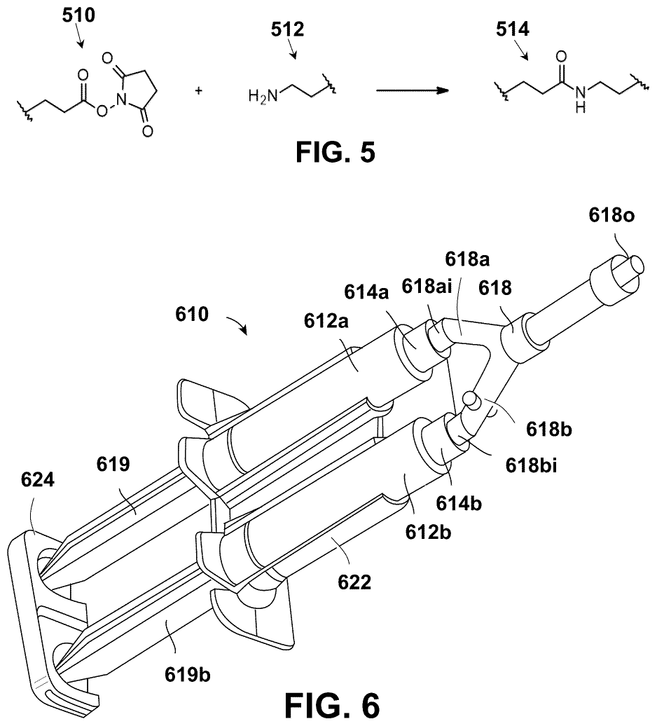

A particular example of a crosslinking reaction is illustrated in FIG. 5, which shows a covalent crosslinking reaction between a cyclic amide ester group, specifically, a succinimide ester group of a radiopaque reactive polymer (510) as described herein and an amino group (512) of a polyamino compound as described herein, whereby an amide linking group (514) is formed.

The radiopaque reactive polymer (510) and the polyamino compound (512) are combined under conditions such that the succinimidyl ester groups of the radiopaque reactive polymer (510) react with the amino groups of the polyamino compound (512) to form amide bonds accompanied by the release of N-hydroxysuccinimide, with the result being a crosslinked polymer network. The crosslinking reaction shown is inhibited at acidic pH, but occurs spontaneously at basic pH. In certain embodiments, reaction between the succinimidyl ester groups and the amino groups is conducted at slightly basic pH (e.g., having a pH value ranging from 7.4 to 11) where the amino groups are deprotonated/neutrally charged and amide bond formation can occur spontaneously at body temperature.

It will be appreciated that the crosslinking density of the radiopaque hydrolysable crosslinked hydrogels described herein can be tuned, for example, by varying the number of amino groups in the polyamino compound, by varying the number of arms of the reactive multi-arm polymer, or both.

In some embodiments, the radiopaque hydrolysable crosslinked hydrogels of the present disclosure are visible using X-ray imaging techniques. The radiopaque hydrolysable crosslinked hydrogels may have a radiopacity that is greater than 100 Hounsfield units (HU), beneficially ranging anywhere from 100 HU to 250 HU to 500 HU to 750 HU to 1000 HU or more (in other words, ranging between any two of the preceding numerical values), for example, when measured on bench-top micro CT systems such as Xtreme CT from Scanco Medical (Wangen-Brüttisellen, Switzerland) or similar.

In some aspects of the present disclosure, a system is provided that comprises (a) a first composition that comprises a polyamino compound, and (b) a second composition that comprises a radiopaque reactive polymer as described herein, wherein the system is configured to deliver the radiopaque reactive polymer and the polyamino compound under conditions such that covalent crosslinks are formed between the radiopaque reactive polymer and the polyamino compound.

The first composition may be a first fluid composition comprising the polyamino compound or a first dry composition that comprises the polyamino compound, to which a suitable fluid such as water for injection, saline, etc. can be added to form a first fluid composition. In addition to the polyamino compound, the first composition may further comprise additional agents, including therapeutic agents, imaging agents, colorants, tonicity adjusting agents, suspension agents, wetting agents, and pH adjusting agents as described below. The second composition may be a second fluid composition comprising the radiopaque reactive polymer or a second dry composition that comprises the radiopaque reactive polymer, to which a suitable fluid such as water for injection, saline, etc. can be added to form a second fluid composition. In addition to the radiopaque reactive polymer, the second composition may further comprise additional agents, including therapeutic agents, imaging agents, colorants, tonicity adjusting agents, suspension agents, wetting agents, and pH adjusting agents as described below.

In some embodiments, the system is configured to combine a first fluid composition comprising the polyamino compound with a second fluid comprising the radiopaque reactive polymer. Upon mixing the first and second fluid compositions, the polyamino compound crosslinks with the radiopaque reactive polymer, forming a crosslinked product. The first and second fluid compositions may be combined to form radiopaque hydrolysable crosslinked hydrogels.

In some embodiments, the polyamino compound is initially combined with the radiopaque reactive polymer under conditions where crosslinking between the radiopaque reactive polymer and the polyamino compound is suppressed (e.g., an acidic pH in the case where the radiopaque reactive polymer comprises reactive electrophilic moieties). Then, when crosslinking is desired, the conditions are changed such that crosslinking is increased (e.g., a change from an acidic pH to a basic pH, in some embodiments), leading to crosslinking between the polyamino compound and the radiopaque reactive polymer, thereby forming a crosslinked product. The first and second fluid compositions may be combined to form radiopaque hydrolysable crosslinked hydrogels.

In some of these embodiments, the system comprises (a) a first composition that comprises a polyamino compound as described hereinabove, (b) a second composition that comprises a radiopaque reactive polymer as described hereinabove, and (c) a third composition, specifically, an accelerant composition, that contains an accelerant that is configured to accelerate a crosslinking reaction between the polyamino compound and the radiopaque reactive polymer.

The first composition may be a first fluid composition comprising the polyamino compound that is buffered to an acidic pH or a first dry composition that comprises the polyamino compound, to which a suitable fluid such as water for injection, saline, an acidic buffer solution, etc. can be added to form a first fluid composition comprising the polyamino compound that is buffered to an acidic pH. In some embodiments, for example, the acidic buffering composition may comprise monobasic sodium phosphate, among other possibilities. The first fluid composition comprising the polyamino compound may have a pH ranging, for example, from about 3 to about 6.5, typically, from about 3 to about 5. In addition to the polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the radiopaque reactive polymer or a second dry composition that comprises the radiopaque reactive polymer from which a fluid composition is formed, for example, by the addition of a suitable fluid such as water for injection, saline, or the first fluid composition comprising the polyamino compound that is buffered to an acidic pH. In addition to the radiopaque reactive polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the polyamino compound that is buffered to an acidic pH and the second composition comprises a dry composition that comprises the radiopaque reactive polymer. The first composition may then be mixed with the second composition to provide a prepared fluid composition that is buffered to an acidic pH and comprises the polyamino compound and the radiopaque reactive polymer. In a particular example, a syringe may be provided that contains the first fluid composition comprising the polyamino compound that is buffered to an acidic pH, and a vial may be provided that comprises the dry composition (e.g., a powder) that comprises the radiopaque reactive polymer. The syringe may then be used to inject the first fluid composition into the vial containing the radiopaque reactive polymer to form a prepared fluid composition that is buffered to an acidic pH and contains the polyamino compound and the radiopaque reactive polymer, which can be withdrawn back into the syringe for administration.

The accelerant composition may be a fluid accelerant composition that is buffered to a basic pH or a dry composition that comprises a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 8.5 to about 12, typically, from about 9 to about 11. In addition to the above, the fluid accelerant composition may further comprise additional agents, including those described below.

A prepared fluid composition that is buffered to an acidic pH and comprises polyamino compound and radiopaque reactive polymer as described above, and a fluid accelerant composition that is buffered to basic pH as described above, may be combined to form radiopaque hydrolysable crosslinked hydrogels.

As previously noted, additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotic agents, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethene (BODIPY) analogs, among others, (e) imageable radioisotopes including 99mTc, 201Th, 51Cr, 67Ga, 68Ga, 111 In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a first fluid composition that comprises a polyamino compound as described herein and a second reservoir that contains a second fluid composition that comprises a radiopaque reactive polymer as described herein, wherein the first and second fluid compositions form a crosslinked product upon mixing.

In some embodiments, the system may include a delivery device that comprises a first reservoir that contains a first fluid composition that comprises a polyamino compound as described herein and a radiopaque reactive polymer as described herein and is buffered to an acidic pH, such as the prepared fluid composition previously described, and a second reservoir that contains second fluid composition, such as the fluid accelerant composition described herein.

In either case, during operation, the first fluid composition and the second fluid composition are dispensed from the first and second reservoirs and combined, whereupon the polyamino compound and the radiopaque reactive polymer and crosslink with one another to form a radiopaque hydrolysable crosslinked hydrogel.

In particular embodiments, and with reference to FIG. 6, the system may include a delivery device 610 that comprises a double-barrel syringe, which includes a first barrel 612a having a first barrel outlet 614a, which first barrel contains a first fluid composition as described above, a first plunger 619a that is movable in the first barrel 612a, a second barrel 612b having a second barrel outlet 614b, which second barrel 612b contains a second fluid composition as described above, and a second plunger 619b that is movable in the second barrel 612b. In some embodiments, the device 610 may further comprise a mixing section 618 having a first mixing section inlet 618ai in fluid communication with the first barrel outlet 614a, a second mixing section inlet 618bi in fluid communication with the second barrel outlet 614b, and a mixing section outlet 6180. Also shown are syringe holder 622 configured to hold the first and second syringe barrels 612a, 612b, in a fixed relationship and a plunger cap 624 configured to hold the first and second plungers 619a, 619b in a fixed relationship. In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a luer connector.

In some embodiments, the delivery device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a radiopaque hydrolysable crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of system that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, in some embodiments, the first and second fluid compositions or a fluid admixture thereof can be injected as a bulking agent, for instance, into tissue around the ureteral orifices for the treatment of vesicoureteral reflux, the first and second fluid compositions or a fluid admixture thereof can be injected for tissue augmentation or regeneration, including cosmetic tissue augmentation, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a radiopaque hydrolysable crosslinked hydrogel is ultimately formed at the administration location.

During and/or after administration, the compositions of the present disclosure can be imaged using a suitable imaging technique. Typically, the imaging technique is an x-ray-based imaging technique, such as computerized tomography or x-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to occlude either a vas deferens or fallopian tube with a crosslinked product of the first and second fluid compositions for the control of reproductive health/family planning, a procedure to implant an embolic composition comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions or fluid admixtures thereof may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, injection for obstruction of the vas deferens, injection for obstruction of the fallopian tube, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

In some embodiments, the radiopaque hydrolysable crosslinked hydrogels are formed ex vivo, in which case the radiopaque hydrolysable crosslinked hydrogels may be in any desired form, including a slab, a cylinder, a coating, or a particle. In some embodiments, the radiopaque hydrolysable crosslinked hydrogel granulated into particles of suitable size. Granulating may be by any suitable process, for instance by grinding (including cryogrinding), homogenization, crushing, milling, pounding, pressing through a screen, or the like. Sieving or other known techniques can be used to classify and fractionate the particles. Hydrogel particles formed using the above and other techniques may vary widely in size, for example, having an average size ranging from 50 to 950 microns.

In addition to a radiopaque hydrolysable crosslinked hydrogel that is formed ex vivo, hydrogel compositions in accordance with the present disclosure may be provided, which contain additional agents, including therapeutic agents, imaging agents, colorants, tonicity adjusting agents, suspension agents, wetting agents, and pH adjusting agents as described above.

In various embodiments, kits are provided that include one or more delivery devices for delivering such hydrogel compositions to a subject. Such systems may include one or more of the following: a syringe barrel, which may or may not contain a hydrogel composition; a vial, which may or may not contain a hydrogel composition; a needle; a flexible tube (e.g., adapted to fluidly connect the needle to the syringe); and an injectable liquid such as water for injection, normal saline or phosphate buffered saline. Whether supplied in a syringe, vial, or other reservoir, the hydrogel composition may be provided in dry form (e.g., powder form) or in a form that is ready for injection, such as an injectable hydrogel form (e.g., a suspension of hydrogel particles).

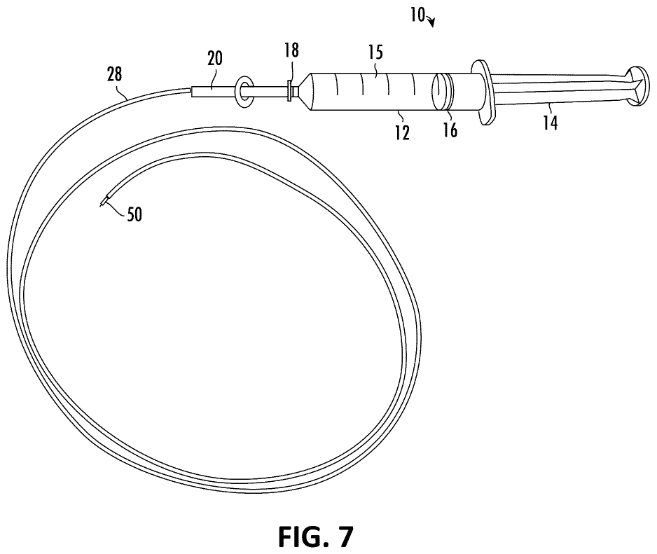

FIG. 7 illustrates a syringe 10 providing a reservoir for a hydrogel composition in accordance with the present disclosure. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a hydrogel composition 15 for injection through the needle 50.

The hydrogel compositions described herein (e.g., a suspension of hydrogel particles, which may also optionally contain additional agents described above) can be used for a number of purposes.

For example, hydrogel compositions can be injected to provide spacing between tissues, hydrogel compositions can be injected (e.g., in the form of blebs) to provide fiducial markers, hydrogel compositions can be injected for tissue augmentation or regeneration, hydrogel compositions can be injected as a filler or replacement for soft tissue, hydrogel compositions can be injected for tissue bulking, hydrogel compositions can be injected for occlusion of lumens, hydrogel compositions can be injected to provide mechanical support for compromised tissue, hydrogel compositions be injected as a scaffold, and/or hydrogel compositions can be injected as a carrier of therapeutic agents in the treatment of diseases and cancers and the repair and regeneration of tissue, among other uses.

During or after administration, the hydrogel compositions of the present disclosure can be imaged using a suitable imaging technique.

As seen from the above, the hydrogel compositions of the present disclosure may be used in a variety of medical procedures, including the following, among others: a procedure to implant a fiducial marker comprising a hydrogel, a procedure to implant a tissue regeneration scaffold comprising a hydrogel, a procedure to implant a tissue support comprising a hydrogel, a procedure to implant a tissue bulking agent comprising a hydrogel, a procedure to occlude a lumen, a procedure to implant a therapeutic-agent-containing depot comprising a hydrogel, a tissue augmentation procedure comprising implanting a hydrogel, a procedure to introduce a hydrogel between a first tissue and a second tissue to space the first tissue from the second tissue, among others.

The hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, injection to increase coaptation of a bodily sphincter such as an anal sphincter for or a urinary sphincter, injection for vas deferens occlusion, injection for fallopian tube occlusion, injection for seminal vessel occlusion, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Hydrogel compositions in accordance with the present disclosure include lubricious compositions for medical applications, compositions for therapeutic agent release (e.g., by including one or more therapeutic agents in a matrix of the hydrogel), and implants (which may be formed ex vivo or in vivo) (e.g., compositions for use as tissue markers, compositions that act as spacers to reduce side effects of off-target radiation therapy, cosmetic compositions, etc.).

It should be understood that this disclosure is, in many respects, only illustrative and that changes may be made in details without exceeding the scope of the disclosure. This may include, to the extent that it is appropriate, the use of any of the features of one embodiment being used in other embodiments.

1. A multi-arm polymer comprising a core region, a plurality of polymer arms linked to the core region, at least three of the polymer arms each comprising a hydrophilic polymer segment and an amino-alcohol residue that is attached to the hydrophilic polymer segment through an ether group, an electrophilic group attached to each amino-alcohol residue through a linkage that comprises a hydrolysable ester group, and a residue of a carboxylic-acid-containing iodinated molecule attached to each amino-alcohol residue through a linkage comprising an amide group.

2. The multi-arm polymer of claim 1, wherein the amino-alcohol residue comprises an amino group and a hydroxyl group on adjacent carbon atoms.

3. The multi-arm polymer of claim 1, wherein the electrophilic group is a cyclic-imidyl ester group.

4. The multi-arm polymer of claim 1, wherein carboxylic-acid-containing iodinated molecule comprises a carboxylic acid group and a monocyclic or multicyclic aromatic structure that is substituted with one or more iodine atoms.

5. The multi-arm polymer of claim 1, wherein the carboxylic-acid-containing iodinated molecule is a carboxyalkylamido-modified, iodinated amino-acid ester, and wherein a residue of an iodinated amino-acid ester is attached to the amino-alcohol residue through a linkage that contains two amide groups.

6. The multi-arm polymer of claim 1, wherein the multi-arm polymer is made by a process comprising (a) reacting the carboxylic-acid-containing iodinated molecule in an amide coupling reaction with a multi-arm polymer comprising the core region and a plurality of polymer arms linked to the core region, at least three of the polymer arms each comprising the hydrophilic polymer segment and an amino-alcohol end group that is attached to the hydrophilic polymer segment through an ether group; (b) reacting the product of step (a) with a cyclic anhydride compound in a ring opening reaction; (c) reacting the product of step (b) in an ester-forming reaction with an N-hydroxy cyclic imide compound.

7. The multi-arm polymer of claim 6, wherein the carboxylic-acid-containing iodinated molecule is made by a process that comprises reacting a cyclic anhydride compound in a ring opening reaction with an amino group of an iodinated amino-acid alkyl ester to form a carboxyalkylamido-modified iodinated amino-acid alkyl ester.

8. A system for forming a hydrogel that comprises (a) the multi-arm polymer of claim 1 and (b) a polyamino compound comprising at least two amino (—NH₂) groups, wherein the reactive multi-arm polymer and the polyamino compound react to form a crosslinked hydrogel.

9. A method of treatment comprising administering to a subject a mixture that comprises the multi-arm polymer of claim 1 and a polyamino compound under conditions such that the multi-arm polymer and the polyamino compound form covalent crosslinks after administration.

10. A multi-arm polymer comprising a core region, a plurality of polymer arms linked to the core region, at least three of the polymer arms each comprising a hydrophilic polymer segment and a residue of a halogen-substituted cyclic anhydride that is attached to the hydrophilic polymer segment through a hydrolysable ester group, an electrophilic group attached to each halogen-substituted cyclic anhydride residue, and at least one residue of a hydroxyl-containing iodinated molecule attached to each halogen-substituted cyclic anhydride residue through a linkage comprising an ether group, wherein the halogen is selected from bromine and iodine.

11. The multi-arm polymer of claim 10, wherein the halogen-substituted cyclic anhydride comprises two or more halogen groups.

12. The multi-arm polymer of claim 10, wherein the electrophilic group is a cyclic-imidyl ester group that is directly attached to a carbon atom of each halogen-substituted cyclic anhydride residue.

13. The multi-arm polymer of claim 10, wherein the hydroxyl-containing iodinated molecule comprises a hydroxyl group and a monocyclic or multicyclic aromatic structure that is substituted with one or more iodine atoms.

14. The multi-arm polymer of claim 10, wherein the multi-arm polymer is made by a process comprising (a) reacting a hydroxyl-terminated multi-arm polymer, which comprises the core region and a plurality of polymer arms linked to the core region, at least three of the polymer arms each comprising the hydrophilic polymer segment and a hydroxyl end group, with a halogen-substituted cyclic anhydride compound to form a multi-arm polymer, which comprises the core region and a plurality of polymer arms linked to the core region, at least three of the polymer arms each comprising the hydrophilic polymer segment and a carboxyhaloalkyl end group that is attached to the hydrophilic polymer segment through a hydrolysable ester group; (b) reacting the product of step (a) with at least one hydroxyl-containing iodinated molecule in a substitution reaction to form a multi-arm polymer, which comprises the core region and a plurality of polymer arms linked to the core region, at least three of the polymer arms each comprising the hydrophilic polymer segment and at least one residue of the hydroxyl-containing iodinated molecule attached to a residue of the carboxyhaloalkyl end group through a linkage comprising an ether group; and (c) reacting the product of step (b) in an ester-forming reaction with an N-hydroxy cyclic imide compound.

15. A multi-arm polymer comprising a core region, a plurality of polymer arms linked to the core region, at least three of the polymer arms each comprising a hydrophilic polymer segment and a residue of a carboxylic-acid-substituted cyclic anhydride that is attached to the hydrophilic polymer segment through a hydrolysable ester group, a residue of a hydroxyl-containing iodinated molecule attached to each carboxylic-acid-substituted cyclic anhydride residue through a linkage that comprises a urethane group and an amide group, and an electrophilic group attached to each carboxylic-acid-substituted cyclic anhydride residue.

16. The multi-arm polymer of claim 15, wherein the electrophilic group is a cyclic-imidyl ester group that is directly attached to a carbon atom of each carboxylic-acid-substituted cyclic anhydride residue.

17. The multi-arm polymer of claim 15, wherein the linkage that comprises a urethane group and an amide group contains a residue of compound that comprises an amine group and an isocyanate group.

18. The multi-arm polymer of claim 15, wherein the carboxylic-acid-substituted cyclic anhydride comprises two or more carboxylic acid groups.

19. The multi-arm polymer of claim 15, wherein the hydroxyl-containing iodinated molecule comprises a hydroxyl group and a monocyclic or multicyclic aromatic structure that is substituted with one or more iodine atoms.

20. The multi-arm polymer of claim 15, wherein the multi-arm polymer is made by a process comprising (a) reacting a hydroxyl-terminated multi-arm polymer, which comprises the core region and a plurality of polymer arms linked to the core region, at least three of the polymer arms each comprising the hydrophilic polymer segment and a hydroxyl end group, in a ring opening reaction with carboxylic-acid-substituted cyclic anhydride compound, in which the carboxylic acid is protected, to form a multi-arm polymer that comprises the core region and a plurality of polymer arms linked to the core region, at least three of the polymer arms each comprising the hydrophilic polymer segment and an end group that comprises the protected carboxylic acid group and an unprotected protected carboxylic acid group; (b) reacting a hydroxyl-containing iodinated molecule with a compound that comprises a protected amine group and an isocyanate group and then deprotecting the amine group to form a molecule in which an amine group is attached to a residue of the hydroxyl-containing iodinated molecule through a urea-containing linkage; (c) reacting the product of step (a) with the product of step (b) in an amide coupling reaction; (d) deprotecting the protected carboxylic acid group; and (e) reacting the product of step (d) in an ester-forming reaction with an N-hydroxy cyclic imide compound.
